(19) United States
(12) Patent Application Publication
(10) Pub. No.: US 2023/0002728 A1
(43) Pub. Date: Jan. 5, 2023

(54) STEM CELL FATE ENGINEERING METHODS

(71) Applicant: Wisconsin Alumni Research Foundation, Madison, WI (US)

(72) Inventors: Sean Palecek, Verona, WI (US); Gyuhyung Jin, Madison, WI (US)

(21) Appl. No.: 17/856,653

(22) Filed: Jul. 1, 2022

Related U.S. Application Data
(60) Provisional application No. 63/217,486, filed on Jul. 1, 2021.

(51) Int. Cl. C12N 5/0735; G01N 33/483; G01N 33/50

(52) U.S. Cl. CPC C12N 5/0606; G01N 33/4833; G01N 33/5044

(57) ABSTRACT

This disclosure relates generally to the differentiation of human pluripotent stem cells, and more particularly to a method of spatially adsorbing morphogens to differentiate human pluripotent stem cells.

### CROSS-REFERENCE TO RELATED APPLICATIONS

This application claims priority to U.S. Provisional Patent Application No. 63/217,486 filed Jul. 1, 2021, which is incorporated herein by reference in its entirety.

### STATEMENT REGARDING FEDERALLY SPONSORED RESEARCH

This invention was made with government support under EB007534 awarded by the National Institutes of Health and under 1648035 and 1743346 awarded by the National Science Foundation. The government has certain rights in the invention.

### FIELD OF DISCLOSURE

This disclosure relates generally to differentiation of human pluripotent stem cells, and more particularly to methods of spatially adsorbing morphogens to a suitable surface upon which human pluripotent stem cells can be differentiated.

### BACKGROUND

During development, embryonic epiblast cells give rise to virtually all types of cells in a human body through precise and robust cell fate determination. This process is governed by elaborate, dynamic spatiotemporal control of physiochemical cell signaling exerted by the cellular microenvironment. Prior art studies have revealed that both soluble or immobilized morphogens control cell signaling and developmental pathways, thereby determining cell fate and patterning (Perrimon et al. (2012) Cold Spring Harb. Perspect. Biol 4.). Spatial regulation of these developmental cues is an important aspect of pattern formation in tissues. Advances in imaging and computational analysis techniques have allowed researchers to investigate in vivo pattern formation induced by morphogens in animal models (Shimozono et. al. (2013) Nature 496, 363-366; Zagorski et al. (2017) Science 356, 1379-1383), but in vivo study of these developmental events in humans remains elusive. In addition, in vivo animal models lack systematic, precise control of morphogens, limiting their use in understanding how the nature of these morphogens regulates cell fate and pattern formation.

The development of human pluripotent stem cells (hPSCs) including human embryonic stem cells (hESCs) and human induced pluripotent stem cells (hiPSCs) has provided a powerful in vitro model not only for studying human development, but also for facilitating drug screening for therapeutics, modeling disease, and developing cell-based regenerative therapies. While temporal modulation of developmental pathways via molecular agonists and antagonists at specific differentiation stages has led to efficient methods to differentiate hPSCs to many different somatic cell types, spatial pattern formation from hPSCs has been more challenging mainly due to difficulties in spatially localizing morphogens in conventional cell culture platforms.

Self-organization of hPSCs or mouse PSCs under specific culture conditions, including different colony sizes, 3D culture, or specialized substrates, has been used to demonstrate early developmental events such as germ layer specification (Warmflash et al. (2014) Nature Methods 11, 847-854; Poh et al. (2014) Nature Comm. 5, 1-12), anteroposterior patterning (Moris et al. (2020) Nature 582, 410-415), and ectodermal patterning (Zheng et al. (2019) Science advances 5; Britton et al. (2019) Development 146). However, this self-assembled patterning inherently lacks regulatory features with regard to spatial control of signaling cues. Alternatively, geometrically confining hPSC colonies into various shapes allowed investigation of effects of producing asymmetrically pre-patterned colonies on hPSC fate (Ma et al. (2015) Nature Comm. 6, 1-10; Smith et al. (2018) Proc. Natl. Acad. Sci. USA 115, 8167-8172). Such cell patterning methods also lack spatial regulation of signaling factors and interaction between patterned and surrounding cells which is critical for proper development in vivo.

Microfluidic devices offer a more complex model that can incorporate both spatial patterning and morphogen gradients. For example, a microfluidic device has been used to create an asymmetric germ layer pattern of hPSC colonies mimicking the presence of proximal morphogen-secreting cells (Manfrin et al. (2019) Nature methods 16, 640). Establishment of human neural tube patterning was also demonstrated in a microfluidic device via a Wnt-activating gradient (Rifes et al. (2020) Nature Biotech. 1-9). However, in addition to the use of specially designed apparatus, this strategy introduces substantial barriers to routine cell culture techniques necessitating specialized substrates, unique methods for media exchange, small medium volumes, and generation of hydrodynamic shear (Halldorsson et al. (2015) Biosens. and Bioelec. 63, 218-231.

Substrate-mediated delivery of signaling factors, primarily through immobilization on a surface, is a conventional strategy to localize inductive factors that mimics natural protein sequestration and presentation in cellular microenvironment (Hettiaratchi et al. (2016) J. of Mat. Chem. 4, 3464-3481). Many of these methods utilize functionalization of substrates to tether signaling molecules thereupon but typically surface functionalization is not spatially controlled and results in uniform substrate modification lacking regional specification (Alberti et al. (2008) Nature methods 5, 645; Pompe et al. (2010) Nature Protocols 5, 1042; Kilian et al. (2012) Angewandte Chemie 124, 4975-4979; Ham et al. (2017) Acta biomaterialia 53, 140-151). Additionally, most approaches to generate patterns require complex strategies, including functionalizing factors (Kim et al. (2012) Angewandte Chemie 124, 5696-5699; Wang et al. (2014) Advanced healthcare materials 3, 214-220) and/or the substrate (Li et al. (2011) PNAS 108, 11745-11750; DeForest et al. (2015) Nature materials 14, 523-531) to be immobilized, or special techniques such as bioprinting (Ker et al. (2011) Biomaterials 32, 3413-3422).

A simpler, more versatile approach to pattern hPSC differentiation is needed in the art.

### SUMMARY OF THE DISCLOSURE

Provided herein are in vitro methods for differentiating pluripotent stem cells, the methods comprising: patterning one or a plurality of signaling proteins on a portion of a suitable surface; coating the suitable surface comprising the one or a plurality of signaling proteins with a cell culture substrate; and plating a suspension of the pluripotent stem cells on the suitable surface coated with the cell culture substrate.

In certain embodiments of the methods of the invention the cell culture substrate is Matrigel®, vitronectin, or laminin. In these and other particular embodiments, the one or plurality of signaling proteins are patterned on the suitable surface at concentrations of 1 ng/μ1 to about 1 μg/μ1. Advantageous surfaces suitable in the practice of the methods of the invention are hydrophilic, including but not limited to a tissue-culture treated polystyrene plate. In particular embodiments the one or a plurality of signaling proteins are placed on the suitable surface in a geometric pattern or are patterned using a polydimethylsiloxane (PDMS) stencil cut into a desired shape or in a concentration gradient. One or a plurality of signaling proteins used in the methods described herein are a morphogen, including but not limited to bone morphogenic protein 4 (BMP4), activin A, noggin, or Wnt3a. As set forth herein the pluripotent stem cells are human embryonic stem cells (hESCs) or human induced pluripotent stem cells (hiPSCs), which can advantageously be is plated at concentrations of about 5×10⁵ to about 1×10⁶ cells/cm′. The practice of the methods provided herein can result in the pluripotent stem cells being differentiated into one or more of mesoderm, endoderm, ectoderm, cardiomyocytes or epicardial cells.

These and other features, objects, and advantages of the invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### BRIEF DESCRIPTION OF DRAWINGS

FIG. 1A-FIG. 1D show localized differentiation of H9 hESCs into BRA+ mesendoderm by BMP4 patterning. FIG. 1A. Immunofluorescence images of BRACHYURY (BRA) at day 2 of differentiation on BMP4-patterned substrate and their quantification in the bar graph at the right of the images. FIG. 1B. Immunofluorescence images for the indicated fate markers. Enlarged immunofluorescence images show the cells on the BMP4-pattern, outside the pattern, and at the pattern boundary. Changes in normalized BRA staining intensity with distance are shown graphically to the right of the images. FIG. 1C. Immunofluorescence images for BRA and MIXL1 at day 2 of differentiation on the BMP4-pattern and outside the pattern. FIG. 1D. Immunofluorescence images for the indicated fate marker after noggin patterning. Scale bars=1 mm, unless otherwise indicated.

FIG. 2A-FIG. 2C show localized differentiation of H9 hESCs into SOX17+ definitive endoderm by activin A patterning. FIG. 2A. Immunofluorescence images of SOX17 at day 3 of the differentiation and their quantification in the bar graph at the right of the images. FIG. 2B. Immunofluorescence images for the indicated fate markers. Activin A was patterned using a heart-shaped PDMS stencil before hESC seeding. Enlarged immunofluorescence images show the cells on the activin A patterns, outside the patterns, and at the pattern boundary. Changes in normalized SOX17 staining intensity with distance are shown graphically to the right of the images. FIG. 2C. Immunofluorescence images for SOX17 and FOXA2 at day 5 of the differentiation. Scale bars=1 mm, unless otherwise indicated.

FIG. 3A-FIG. 3D shows localized multi-lineage differentiation of H9 hESCs into TE7+ mesoderm cells and SOX17+ endoderm cells on BMP4 and activin A patterns. FIG. 3A. Immunofluorescence images for the indicated fate markers at day 7 of differentiation and their quantification for each in the bar graph at the right of the images. FIG. 3B. Immunofluorescence images for ectodermal markers OTX2 and SOX2 on region outside of the BMP4 and activin A patterns at day 7 of differentiation. Immunofluorescence images at the boundary of FIG. 3C. BMP4 and FIG. 3D. activin A patterns. Scale bars=1 mm, unless otherwise indicated. Changes in normalized staining intensity with distance for SOX17, SOX2, and TE7 are shown graphically to the right of the images.

FIG. 4A-FIG. 4D shows localized differentiation of H9 and H9-hTNNT2-GFP hESCs into cTnT+ CMs on BMP4 patterns. FIG. 4A. Schematic diagram of the CM differentiation protocol. BMP4 patterns drive mesoderm induction and Wnt inhibition directs cardiac mesoderm induction. FIG. 4B. Immunofluorescence images for cTnT in CMs differentiated from H9 hESCs (2.5 ng/μl BMP4 patterned by a circle-shaped PDMS stencil on a TC-treated plate prior to washing, Matrigel® coating, and hESC seeding) and H9-hTNNT2-GFP hESCs (2.5 ng/μl BMP4 pattern by a star-shaped PDMS stencil on a TC-treated plate prior to washing, Matrigel® coating, and hESC seeding). FIG. 4C. Immunofluorescence images for cTnT (CM marker) and SOX2 (ectoderm cell marker) in H9 hESC-derived CMs and their quantification (2.5 ng/μl BMP4 patterned by a heart-shaped PDMS stencil on a TC-treated plate prior to washing, Matrigel coating, and hESC seeding) and surrounding cells shown in the bar graph at the right of the images. Enlarged immunofluorescence images show cells on the BMP4-pattern, outside the pattern, and at the boundary of the pattern. Changes in normalized staining intensity with distance for cTnT SOX2 and TE7 are shown graphically to the right of the images. FIG. 4D. Immunofluorescence images for OTX2 and SOX2 (ectoderm cell markers), and PAX6 (neuroectoderm marker) in the cells outside of the BMP4 pattern. Nuclear localization of PAX6 is indicated by white arrows. Scale bars=1 mm, unless otherwise indicated.

FIG. 5A-FIG. 5C shows localized differentiation of H9 hESC-derived cardiac progenitor cells (CPCs) into WT1+ EpiCs and cTnT+ CMs by patterning Wnt3a and BMP4 adsorption. FIG. 5A. Immunofluorescence images for the indicated fate markers 7d after cardiac progenitor seeding on morphogen patterns. FIG. 5B. Immunofluorescence images for cTnT (CM marker) and WT1 (EpiC marker) and their quantification shown in the bar graph at the right of the images. Wnt3a (40 ng/μl)+BMP4 (2 ng/μl) solution was placed in a circle-, heart-, or star-shaped PDMS stencils before Matrigel coating and cardiac progenitor seeding. Enlarged immunofluorescence image at the boundary of the pattern shows the two distinct cell fates (CM and EpiC) and its transition along the pattern edge. Changes in normalized staining intensity with distance for cTnT and WT1 are shown graphically to the right of the images. FIG. 5C. Immunofluorescence images for cTnT (CM marker) and WT1 (EpiC marker). A heart-shaped PDMS mask was placed at the center of the well to exclude Wnt3a and BMP4 adsorption from the heart-shaped region upon application of Wnt3a (40 ng/μl)+BMP4 (2 ng/μl) solution prior to washing, Matrigel® coating, and cardiac progenitor seeding. Scale bars=1 mm, unless otherwise indicated.

FIG. 6A-FIG. 6C shows the effects of EpiCs on CM structure during localized codifferentiation on BMP4 and Wnt3a-patterned substrates. FIG. 6A. Epifluorescence images of α-actinin and F-actin immunostaining showing the alignment of CM sarcomeres and myofibril filaments at the boundary of the BMP4 and Wnt3a pattern (indicated by arrow). FIG. 6B. Confocal fluorescence images for α-actinin, MLC2a (CM marker) and WT1 (EpiC marker) showing α-actinin staining at the edge of the patterns. The maximum intensity projection of multiple z-slices and the side view of these z-slices shows the spatially organized CMs and EpiCs (EpiCs indicated by arrows). FIG. 6C. Quantification for the length and the alignment of sarcomeres. The statistical comparison was performed using a one-way ANOVA with Tukey's host hoc test (*** P<0.001, n=3). Error bars represent standard deviations of the means of 500 lengths and 25 angles across three biological replicates for each condition. Scale bars are indicated in each image.

FIG. 7A-FIG. 7C shows localized patterning of BSA-FITC. FIG. 7A. Fluorescence images for BSA-FITC patterning. 100 ng/μl BSA-FITC solution was placed as a droplet (Droplet), by pipette drawing (Drawing), or with PDMS stencil masks (Stencil) on untreated or TC-treated plates, incubated for 30 min at room temperature, and washed before imaging. FIG. 7B. Fluorescence images for BSA-FITC adsorbed from solutions with the indicated concentrations on untreated PS or TC-treated PS plates. FIG. 7C. Fluorescence images for 100 ng/μl BSA-FITC at different incubation times. Localized patterning of BSA-FITC was performed independently at least three times with similar results. Scale bars=1 mm.

FIG. 8A-FIG. 8B shows localized patterning of BMP4. FIG. 8A. Immunofluorescence images for BMP4 patterning. 2.5 ng/μl BMP4 solution was placed as a droplet (Droplet), by pipette drawing (Drawing), or with PDMS stencil masks (Stencil) on TC-treated plates, incubated for 30 min, and washed before staining. FIG. 8B. Immunofluorescence images for BMP4 adsorbed from solutions with the indicated concentrations on untreated PS or TC-treated PS plates. The statistical comparison was performed using two-tailed Student's t test (*** P<0.001, n=4) between the untreated and TC-treated condition at each concentration are shown graphically below the images. Error bars represent standard deviations of four fluorescence intensities of four droplets per condition. Localized patterning of BMP4 was performed independently at least three times with similar results. Scale bars=1 mm.

FIG. 9A-FIG. 9D shows localized differentiation of H9 hESCs into BRA+ mesendoderm on untreated and TC-treated PS plates on BMP4 patterns. FIG. 9A. Untreated PS plates and on FIG. 9B. TC-treated PS plates. Immunostaining for laminin on BMP4 patterns with corresponding BMP4 concentrations on FIG. 9C. untreated PS plates and on FIG. 9D. TC-treated PS plates. BMP4 solution was incubated on the substrates as droplets with the indicated BMP4 concentration for 30 min, then washed before Matrigel® coating. Scale bars=1 mm.

FIG. 10A-FIG. 10B shows localized differentiation of H9 hESCs into BRA+ mesendoderm or SOX17+ definitive endoderm on BMP4 and activin A patterns in the presence of ALK inhibitors. FIG. 10A. Immunofluorescence images for the mesendoderm marker BRACHYURY at day 2 of differentiation. FIG. 10B. Immunofluorescence images for the definitive endoderm marker SOX17 at day 3 of differentiation. Scale bars=1 mm unless otherwise indicated.

FIG. 11A-FIG. 11B shows localized differentiation of H9 hESCs into BRA+ mesendoderm on BMP4 patterns on different substrates (glass and TC-treated ibidi polymer) or with different ECM coatings (Synthemax and vitronectin). FIG. 11A. Immunofluorescence images for BRA at day 2 of differentiation on glass and TC-treated polymer (ibidi polymer). FIG. 11B. Immunofluorescence images for BRA at day 2 of differentiation on Synthemax and vitronectin-coated plates. Scale bars=1 mm unless otherwise indicated.

FIG. 12A-FIG. 12D shows localized differentiation of WTC11 hiPSCs into BRA+ mesendoderm or SOX17+ definitive endoderm on BMP4 and activin A patterns. FIG. 12A. Immunofluorescence images for the mesendoderm marker BRACHYURY at day 2 of differentiation. FIG. 12B. Immunofluorescence images for the indicated fate markers. FIG. 12C. Immunofluorescence images for the definitive endoderm marker SOX17 at day 3 of differentiation. FIG. 12D. Immunofluorescence images for the indicated fate markers. Scale bars=1 mm.

FIG. 13A-FIG. 13B shows localized multi-lineage differentiation of H9 hESCs into MESP1+, TE7+ mesodermal cells and SOX17+ endodermal cells on BMP4 and activin A patterns. FIG. 13A. Immunofluorescence images for the indicated fate markers at day 3 and FIG. 13B. day 5 of the differentiation. The normalized intensity profiles were obtained from the average fluorescence intensity values of SOX17, SOX2, MESP1, and TE7 along the y-axis divided by the average fluorescence intensity value of Hoechst along the y-axis of a representative image at each pattern boundary. Changes in normalized staining intensity with distance for SOX17, SOX2, and MESP1 (FIG. 13A) and for SOX17, SOX2, and TE7 (FIG. 13B) are shown graphically to the right of the images. Scale bars=1 mm unless otherwise indicated.

FIG. 14A-FIG. 14C shows localized differentiation of H9-hTNNT2-GFP line-derived cardiac progenitors into WT1+ EpiCs and cTnT+ CMs on Wnt3a and BMP4-patterned substrates. FIG. 14A. Time-lapse live cell imaging during the patterned differentiation of cardiac progenitors into EpiCs and CMs. FIG. 14B. Immunofluorescence images for the cTnT (CM marker) and WT1 (EpiC marker) 7d after cardiac progenitor seeding. FIG. 14C. Immunofluorescence images for the cTnT (CM marker) and TBX18 (EpiC marker) 7d after cardiac progenitor seeding. Scale bars=1 mm unless otherwise indicated.

### DETAILED DESCRIPTION OF THE DISCLOSURE

All publications, including but not limited to patents and patent applications, cited in this specification are herein incorporated by reference as though set forth in their entirety in the present application.

As utilized in accordance with the present disclosure, unless otherwise indicated, all technical and scientific terms shall be understood to have the meaning commonly understood by one of ordinary skill in the art. Unless otherwise required by context, singular terms shall include the plural and plural terms shall include the singular.

The present disclosure relates to the inventors' straightforward and versatile method to pattern pluripotent stem cell (PSC) differentiation via localized adsorption of substrate-bound morphogens. As demonstrated in this disclosure human PSC (hPSC) differentiation can be spatially regulated by localized patterning of specific morphogens on a suitable substrate.

As used herein, the skilled worker will understand that the term “morphogen” encompasses any molecule that directs cell fate. Signaling proteins can be morphogens but can also have effects broader than cell fate. The signaling proteins used in embodiments of the invention herein are also morphogens. The skilled worker in the art will understand that the term morphogen is broader than the term signaling protein, wherein the effects of morphogens include differentiation, proliferation, changes in metabolism, activation, and like biological activities. The skilled worker will appreciate when molecules described by either term are used in each such particular situation or embodiment of the invention.

In particular embodiments provided herein are in vitro methods for differentiating pluripotent stem cells, the methods comprising: patterning one or a plurality of signaling protein on a portion of a suitable surface; coating the suitable surface comprising the one or a plurality signaling proteins with a cell-culture substrate; and plating a solution of the pluripotent stem cells on the suitable surface coated with the cell-culture substrate.

As used herein, “pluripotent stem cells” are cells having the capacity to differentiate into cells of all three germ layers. Pluripotent stem cells (PSCs) suitable for the differentiation methods disclosed herein include, but are not limited to, human embryonic stem cells (hESCs), human induced pluripotent stem cells (hiPSCs), non-human primate embryonic stem cells (nhpESCs), and non-human primate induced pluripotent stem cells (nhpiPSCs).

As used herein, the term “embryonic stem cells” or “ESCs” means a pluripotent cell or population of pluripotent cells derived from an inner cell mass of a blastocyst. See Thomson et al. (1998) Science 282:1145-1147. These cells can be characterized as expressing Oct-4, SSEA-3, SSEA-4, TRA-1-60, and TRA-1-81, and appear as compact colonies having a high nucleus-to-cytoplasm ratio and prominent nucleolus. ESCs are commercially available from sources such as WiCell Research Institute (Madison, Wis.).

As used herein, the term “induced pluripotent stem cells” or “iPS cells” or “iPSCs” means a pluripotent cell or population of pluripotent cells that are produced in vitro from non-pluripotent cells such as differentiated somatic cells by inducing expression of certain potency-determining factors therein, that can vary with respect to their differentiated somatic cell of origin, that can vary with respect to a specific set of potency-determining factors, and that can vary with respect to culture conditions used to isolate them, but nonetheless are substantially genetically identical to their respective differentiated somatic cell of origin and display characteristics similar to higher potency cells, such as ESCs, as described herein. See, e.g., Yu et al. (2007) Science 318, 1917-1920. Induced pluripotent stem cells (iPSCs) exhibit morphological properties (e.g., round shape, large nucleoli, and scant cytoplasm) and growth properties (e.g., doubling time of about seventeen to eighteen hours) akin to ESCs. In addition, iPS cells express pluripotent cell-specific markers (e.g., Oct-4, SSEA-3, SSEA-4, Tra-1-60, or Tra-1-81, but not SSEA-1). Induced pluripotent stem cells, however, are not immediately derived from embryos. As used herein, “not immediately derived from embryos” means that the starting cell type for producing iPS cells is a non-embryonic, non-pluripotent cell, such as a multipotent cell or terminally differentiated cell, such as somatic cells obtained from a post-natal individual.

In particular embodiments, the disclosed methods employ pluripotent stem cells that are human embryonic stem cells (hESCs) or human induced pluripotent stem cells (hiPSCs).

In particular embodiments, the solution of pluripotent stem cells is plated on the suitable substrate at a concentration of about 5×10⁵ to about 1×10⁶ cells/cm².

As used herein “suitable surface” refers to a surface which allows stem cells to attach and grow. In particular embodiments, the suitable surface is hydrophilic. In further embodiments the hydrophilic surface can be a tissue-culture treated polystyrene plate, a round cover glass, or a μ-dish with a polymer coverslip bottom. In particular embodiments, the suitable surface comprises tissue-culture treated polystyrene plates.

In a particular embodiment, the one or a plurality of proteins are a morphogen. A number of morphogens can be used to differentiate hPSCs. In one embodiment, bone morphogenic protein 4 (BMP4) is patterned to differentiate hPSCs to definitive mesendoderm. In a further embodiment, mesendodermal cells can further be differentiated into cardiomyocytes by adding IWP2 to the culture medium after BMP4 patterning. In another embodiment, cardiac progenitors can be differentiated into epicardial cells by patterning BMP4 with Wnt3a. In another embodiment, activin A is patterned to differentiate hPSCs to definitive endoderm. Other embodiments include combining BMP4 and activin A in the same well for localized multi-lineage differentiation of hPSCs. In particular embodiments, the one or a plurality of proteins can be selected from BMP4, activin A, noggin, or Wnt3a.

In particular embodiments, the pluripotent stems cells are differentiated into one or more of mesendoderm, endoderm, cardiomyocytes or epicardial cells.

As used herein “cell culture substrate” refers to any extracellular matrix suitable to enhance stem cell attachment and survival on a surface. A cell culture substrate can be naturally-occurring or synthetic. In particular embodiments, the cell culture substrate is Matrigel®, vitronectin, or laminin.

The one or a plurality of signaling proteins used in the methods disclosed herein can be placed on the suitable surface as droplets, patterns drawn with a pipette, or confined using a polydimethylsiloxane (PDMS) stencil. In particular embodiments, the one or a plurality of proteins are placed on the suitable surface in a pattern. In other embodiments, the pattern is created on the suitable surface using a PDMS stencil cut into a desired shape. In other embodiments, the one or a plurality of proteins are patterned in a concentration gradient. These straightforward methods to create patterns are broadly accessible, but strategies such as a photolithography microfabrication to create more elaborate PDMS stencils can be applied to pattern protein adsorption on surfaces.

In particular embodiments, the one or a plurality of proteins are patterned on the suitable surface at a concentration of 1 ng/μl to about 1 μg/μl.

The disclosed methods for localized differentiation of hPSCs and committed progenitors provide numerous opportunities to investigate various aspects of developmental events and intercellular interaction in a spatially controlled manner due to its versatility. Unlike other strategies for patterning morphogens, this approach does not require complex functionalization of substrates or morphogen itself nor an additional device such as a bioprinter or a microfluidic device, making it more feasible in biological laboratories. The methods of the invention can advantageously be used for, constructing precisely patterned tissues for screening the effects of drugs and other compounds on tissue structure and function, as a tool to study cell interaction in a spatially controlled manner, and for building patterned micro-tissues for cell-based therapies. Additionally, the practice of this invention does not introduce hydrodynamic shear to cells, thus allowing the study of localized paracrine effects of differentiating cells, one of the key aspects of development.

### Examples

The Examples that follow are illustrative of specific embodiments of the disclosure, and various uses thereof. They are set forth for explanatory purposes only and should not be construed as limiting the scope of the disclosure in any way.

### Example 1: Protein Patterning

To observe whether patterned adsorption can be applied to protein morphogens, bone morphogenetic protein 4 (BMP4) solution was patterned as droplets, drawn letters, or stencil masks and then BMP4 was visualized by immunofluorescent microscopy.

A. Stock Solutions

Stock solution for each protein was prepared as follows: 500 ng/μl BSA-FITC (Invitrogen, A23015) in sterile-filtered water (Sigma, W3500), 10 ng/μl human recombinant BMP4 (Invitrogen, PHC9534) in sterile-filtered 4 mM HCl containing 0.1% human serum albumin (HSA) (Sigma, A9731), 100 ng/μl human recombinant activin A (R&D Systems, 338-AC-010) in sterile-filtered 4 mM HCl, 250 ng/μl human recombinant noggin (R&D Systems, 6057-NG-025) in phosphate buffered saline (PBS) (Gibco, 14190-144) containing 0.1% HSA, and 200 ng/μl human recombinant Wnt3a (R&D Systems, 5036-WN-010) in PBS containing 0.1% HSA. Further dilutions were performed in water for BSA-FITC and PBS for other proteins to the desired final concentrations.

B. Protein Droplets

Diluted protein solution was placed as a 5 μl droplet, drawn with a pipette tip (10 μl for each letter), or confined with PDMS stencil masks (20 μl for each shape) on a TC-treated plate (Corning, 3513 (12-well plate)/3526 (24-well plate)), untreated PS plate (Corning, 351143 (12-well plate)), or 15 mm round cover glass (Electron Microscopy Sciences, 71887-05) placed in a 24-well TC-5 mm μ-dish with a polymer coverslip bottom (ibidi, 81156). To create an inverse pattern, a heart-shaped PDMS mask was placed at the center of the well of a 24-well plate to exclude Wnt3a and BMP4 adsorption from the heart-shaped region upon application of Wnt3a+BMP4 solution in the well.

C. Polydimethylsiloxane (PDMS) Stencil Masks

To make PDMS stencil masks, a PDMS layer was prepared, cut, and punched with commercially available hole punches. Briefly, base elastomer and curing agent (Electron Microscopy Sciences, 24236-10) were mixed thoroughly with a weight ratio of 10:1, poured in a unstick baking pan to cover the surface, de-gassed in a desiccator until bubbles were removed and cured in an oven for an hour at 100° C. and finally cooled for at least 24 hours at room temperature. The cured PDMS layer was detached from the pan and used to make stencil masks. The PDMS masks were firmly attached on substrates to ensure solution confinement. Protein solutions were incubated for 30 min at room temperature and aspirated. Protein solutions containing Wnt3a were incubated for 20 min at room temperature to minimize solution spreading. Immediately after aspiration, substrate was washed with PBS three times and further experiments were performed.

D. Localized Protein Patterns by Confinement of Protein Solutions

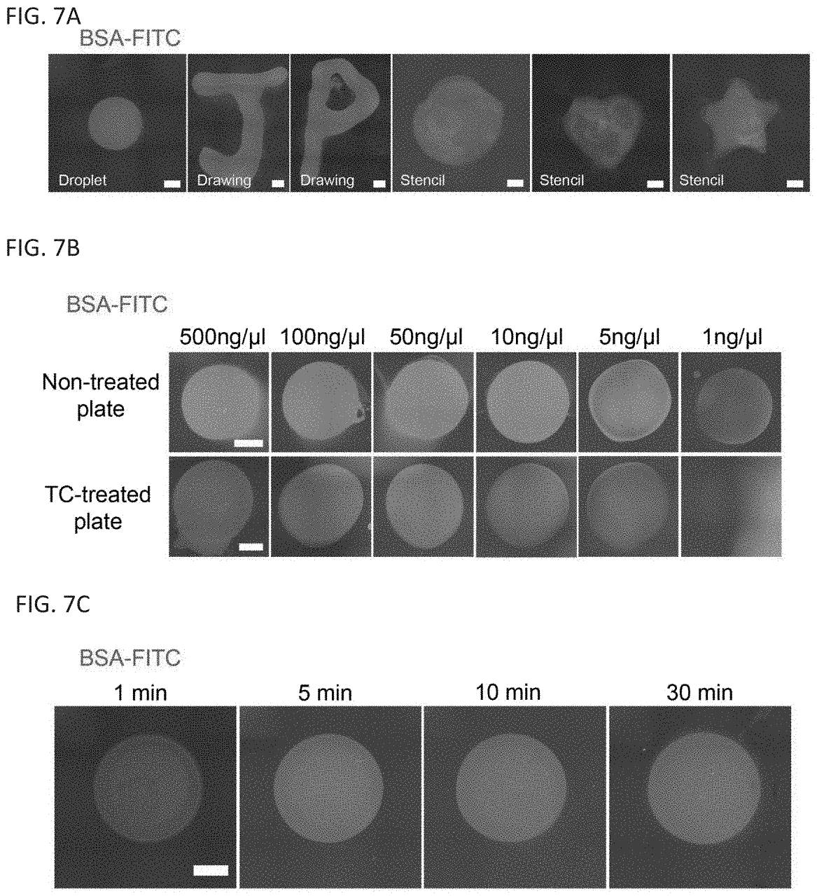

Proteins adsorbed on substrates can mediate cellular responses such as proliferation, migration, and differentiation and they can also drive pattern formation (Hettiaratchi et al. (2016) J. of Mats. Chem. 4, 3464-3481). Placing a protein solution on a substrate generated localized protein adsorption. Bovine serum albumin conjugated with fluorescein isothiocyanate (BSA-FITC) solution was placed on a tissue culture plate as a droplet, drawn with a pipette tip (letter patterning), or confined with polydimethylsiloxane (PDMS) stencil masks (circle, heart, and star patterning). The BSA-FITC remained adsorbed after a washing step while maintaining the patterns (as shown in FIG. 7A). Importantly, more adsorption of BSA-FITC was observed on a hydrophobic untreated polystyrene (PS) plate when compared to the adsorption of BSA-FITC on a tissue culture (TC)-treated PS plate, suggesting that the adsorption likely involves hydrophobic interaction between the protein and the substrate (FIG. 7B). Then, the effects of the different incubation time on adsorption were assessed (FIG. 7C). Even a short incubation (1 min) of the droplet resulted in a localized adsorption, suggesting that the interaction is rapid, consistent with the time scale of a hydrophobic interaction between a surfactant and a mineral surface (Meyer et al. PNAS 103, 15739-15746).

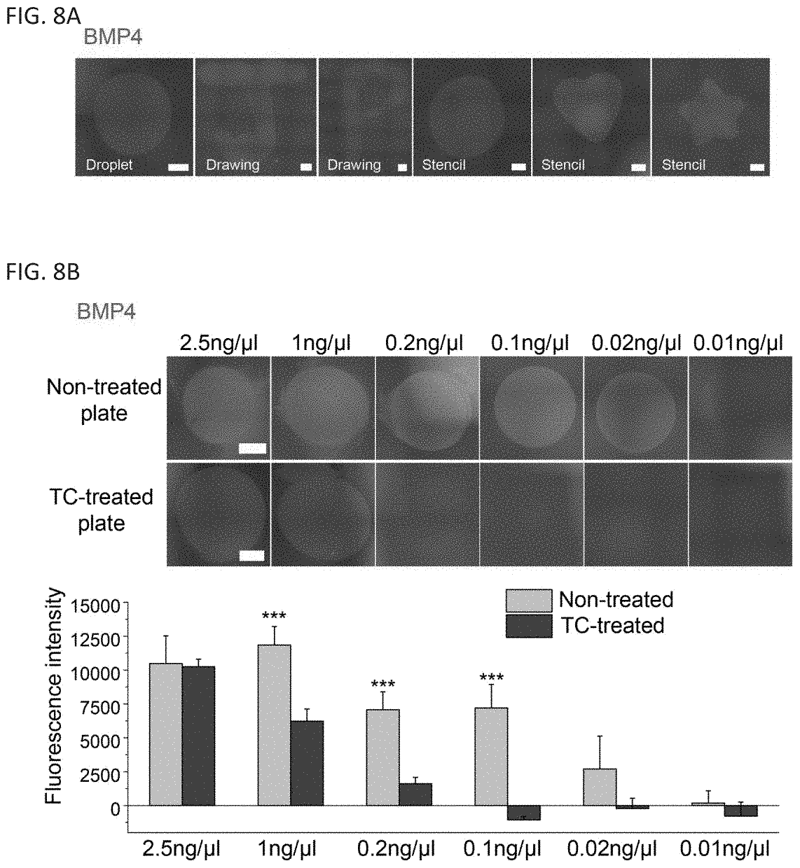

Like BSA-FITC, BMP4 retained its patterns after washing (FIG. 8A) and more BMP4 was adsorbed on the hydrophobic untreated plate than the TC-treated plate (FIG. 8B). These data demonstrated that a localized protein pattern can be created via a simple placement and confinement of a protein solution due to adsorption of the protein on the substrate.

### Example 2: hPSC Maintenance and CPC Differentiation

A. hPSC Maintenance

H9 hESC (WiCell), H9-hTNNT2-GFP hESC (H9-hTnnT2-pGZ-TD2, WiCell), and WTC11 (Allen Institute) were maintained in mTeSR1 basal medium (STEMCELL Technologies, 85851) supplemented with 5× supplement (STEMCELL Technologies, 85852). When cells reached 70-80% confluency, they were passaged onto a Matrigel® (Corning, 354230)-coated 6-well TC-treated plate (Corning, 3516) with a split ratio of 1:9 using Versene (Gibco, 15040-066) (6 min incubation at 37° C.). Matrigel®-coated plates were prepared by diluting Matrigel® in DMEM/F12 medium (Gibco, 11330-032) to a concentration of 0.08 mg/ml, adding the Matrigel® solution to TC-treated plates, and incubating the plates at least 2 hours at 37° C.

B. Cardiac Progenitor Cell (CPC) Differentiation

CPCs were generated using the GiWi protocol (Bao et al. (2016) Nature Biomed. Eng. 1, 1-12). Briefly, hPSCs were singularized using Accutase (Innovative Cell Technologies, AT104) (10 min incubation at 37° C.), resuspended in mTeSR1 supplemented with 5 μM ROCK inhibitor Y-27632 (Selleckchem, S1049), and seeded at 250,000-400,000 cells/cm² on a Matrigel®-coated 12-well TC-treated plate (Corning, 3513) (day −2). On the next day, the medium was changed with fresh mTeSR1. At day 0, cells were treated with 9-12 μM CHIR99021 (Selleckchem, 51263) in RPMI1640 medium (Gibco, 11875-093) containing 1× B27 supplement minus insulin (A18956-01) (RPMI B27−) for 24 hours. At day 1, medium was changed with fresh RPMI B27−. At day 3, cells were treated with 5 μM IWP2 (Tocris, 3533) in fresh RPMI B27−. At day 5, medium was changed with fresh RPMI B27−. At day 6, cells were detached using Accutase (10 min incubation at 37° C.) and cryopreserved in a cryopreservation medium of 60% RPMI1640 medium containing 1× B27 supplement (17504-044) (RPMI B27+), 30% FBS (Peak Serum, PS-FB1), 10% dimethyl sulfoxide (DMSO) (Sigma, D2650), and 5 μM Y-27632.

### Example 3: Patterned BMP4

A. Localized Mesendoderm Differentiation of hPSCs

For localized mesendoderm differentiation, 2-2.5 ng/μl BMP4 solution was placed with various patterns on different substrates. For glass surfaces, 1.5 ng/μl BMP4 solution was used. 5 ng/μl noggin solution was placed as a droplet to show an inhibitory effect of noggin on BMP4-induced mesendoderm differentiation. After washing, substrate was coated with Matrigel® for 2 hours as described above. Glass was coated with Matrigel® overnight. 0.025 mg/ml Synthemax (Corning, 3535) (final concentration, diluted in water) was added to a washed TC-treated plate and the plate was incubated for 2 hours at room temperature prior to cell seeding. 5 μg/ml truncated recombinant human vitronectin (Gibco, A14700) diluted in PBS was added to a washed TC-treated plate and the plate was incubated for 1 hour at room temperature prior to cell seeding. After ECM coating, the ECM solution was aspirated and hPSCs were singularized using Accutase (10 min incubation at 37° C.), resuspended in mTeSR1 supplemented with 5 μM Y-27632, and seeded at 5×10⁵ to about 1×10⁶ cells/cm² on coated substrates (day 0). On the next day (day 1), medium was changed with fresh mTeSR1. When the noggin solution was placed as a droplet, mTeSR1 supplemented with 10 ng/ml BMP4 was used as medium at day 0 and day 1. For the actin-receptor like kinases (ALK) inhibitor test, 1 μM A83-01 (Tocris, 2939), 10 μM SB-431542 (Tocris, 1614), or 2 μM LDN-212854 (Tocris, 6151) was added to mTeSR1 at day 0. At day 2, cells were immunostained for analysis.

B. Immunofluorescence

For BMP4 staining, Alexa Fluor™ 488 Tyramide SuperBoost™ Kit (Invitrogen, B40941) was used according to the manufacturer's instructions. Briefly, BMP-patterned plates were incubated in 10% goat serum for 1 hour at room temperature and incubated with BMP4 antibody (OriGene, TA500014) diluted in 10% goat serum (1:100 dilution) overnight at 4° C. On the next day, the surface was rinsed with PBS three times and incubated in poly-horseradish peroxidase (HRP)-conjugated secondary antibody overnight at 4° C. Again, the surface was rinsed with PBS three times and incubated in the Tyramide working solution for 8 min at room temperature. Then, the same volume of Reaction Stop Reagent was added to the surface followed by washing three times with PBS before observation under the fluorescence microscope.

C. Immunostaining

For immunostaining of other protein markers, cells were fixed in 4% paraformaldehyde (Electron Microscopy Sciences, 15710-S) in PBS for 15 min at room temperature, washed with PBS three times, and incubated in diluted primary antibodies in 5% nonfat dry milk (Santa Cruz Biotechnology, sc-2324)/0.4% Triton X-100 (Fisher Scientific, BP151-500) PBS overnight at 4° C. Then, the cells were washed with PBS three times and incubated in diluted secondary antibody in 5% nonfat dry milk/0.4% Triton X-100 PBS for 30 min at room temperature. After secondary antibody staining, the cells were washed with PBS three times, incubated in 2 μg/ml Hoechst 33342 diluted in PBS for 5 min at room temperature, washed with PBS two times, and analyzed under the fluorescence microscope.

D. Primary Antibodies

Primary antibodies and corresponding dilution ratio were listed as follows: anti-BMP4 (OriGene, TA500014/1:100), anti-BRACHYURY (R&D Systems, AF2085/1:100), anti-MIXL1 (Proteintech, 22772-1-AP/1:100), anti-Laminin (Novus Biologicals, NB300-144/1:100), anti-SOX17 (R&D Systems, AF1924/1:100), anti-SOX2 (Invitrogen, 14-9811-82/1:100), anti-NANOG (Cell Signaling Technology, 4893S/1:1000), anti-FOXA2 (Invitrogen, 720061/1:500), anti-MESP1 (R&D Systems, MAB9219-100/1:100), anti-TE7 (EMD Millipore, CBL271/:100), anti-OTX2 (R&D Systems, AF1979/1:100), anti-cTnT (Invitrogen, MA5-12960/1:200), anti-PAX6 (EMD Millipore, AB2237/1:250), anti-WT1 (Invitrogen, MA5-32215/1:100), anti-TBX18 (Abcam, ab115262/1:200), anti-α-actinin (Sigma, A7811/1:1000), anti-MLC2a (Synaptic Systems, 311011/1:200), and anti-F-actin (pre-conjugated to DyLight 594) (Invitrogen, 21836/1:50).

E. Secondary Antibodies

Secondary antibodies and corresponding dilution ratio were listed as follows: donkey anti-goat IgG Alexa Fluor 488 (Invitrogen, A11055/1:1000), goat anti-mouse IgG1 Alexa Fluor 488 (Invitrogen, A21121/1:1000), chicken anti-rabbit IgG Alexa Fluor 488 (Invitrogen, A21441/1:1000), donkey anti-rat IgG Alexa Fluor 555 (abeam, ab150154/1:1000), goat anti-mouse IgG2b Alexa Fluor 555 (Invitrogen, A21147/1:1000), donkey anti-mouse IgG1 Alexa Fluor 647 (Invitrogen, A21240/1:1000), and donkey anti-rabbit IgG Alexa Fluor 647 (Invitrogen, A31573/1:1000).

F. Image Analysis

Fluorescence intensity of BMP4 was quantified using Fiji/ImageJ software. From gray scale images, mean gray value of BMP4-patterned region was subtracted by mean gray value of the background. Four independent droplets for each concentration were quantified. The statistical comparison was performed using two-tailed Student's t test (** P<0.01) between untreated and TC-treated conditions at each concentration. Intensity profiles at pattern boundaries were obtained using Fiji/ImageJ software. A representative image at each pattern boundary was transformed into gray scale images, then vertically averaged pixel intensities for all pixels along the x-axis were obtained for each marker. Averaged intensities for each marker were divided by the corresponding averaged intensities of nuclei and plotted to get normalized intensity profiles. Maximum intensity projection images of z-stack images were obtained using NIS-Elements imaging software (NIS-Elements Advanced Research version 5.20.01). For analysis of length and alignment of sarcomeres, 25 images for each condition were obtained from three biological replicates. The images were blinded using an ImageJ plugin ‘Blind Analysis Tools’ generating encrypted file names with a mapping file. For each image, 20 lines across 4 α-actinin+ z-lines were quantified (500 lines per each condition) using NIS-Elements imaging software to get the lines' lengths and positional information. From the positional information of all 20 lines, angles between those lines were calculated. Specifically, a line was chosen as a reference line to obtain 19 different angles between the reference line and the other lines. These 19 values were averaged to get an angle difference value of a single line. The same calculation was performed for all 20 lines to get 20 different angles. These 20 angles were averaged again to get a representative value for angle between sarcomeres of a single image. The 25 angles from 25 images of each condition were averaged and standard deviations were calculated to get the reported values. The statistical comparison was performed with 500 lengths or 25 angles per each condition using a one-way ANOVA with Tukey's host hoc test (*** P<0.001, n=3)

G. Localized Differentiation of hPSCs into Mesendoderm by Patterned BMP4

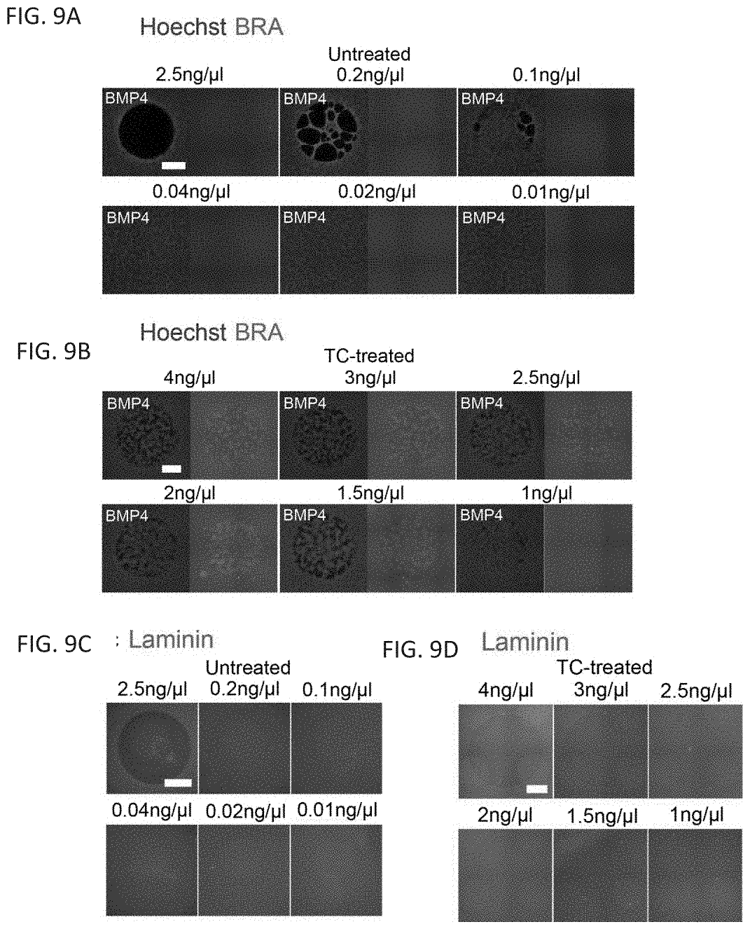

To explore whether spatial localization of adsorbed morphogens can mediate pattern formation during hPSC differentiation, different concentrations of BMP4 droplets were placed on either untreated PS or TC-treated PS plates followed by Matrigel® coating and subsequent plating of hPSCs. The cells were seeded to generate a confluent monolayer. BMP4 in the presence of fibroblast growth factor 2 (FGF2) is known to induce primitive-streak like mesendoderm specification of hPSCs, characterized by expression of BRACHYURY (BRA) (Yu et al. (2011) Cell stem cell 8, 326-334). While none of the BMP4 droplets induced mesendoderm differentiation on the untreated PS surface after two days of culture in the hPSC maintenance medium mTeSR1, a range of BMP4 concentrations induced localized mesendoderm differentiation on TC-treated PS (shown in FIG. 9A and FIG. 9C). This outcome can be attributed to the hydrophobic interaction between BMP4 and the surface, which can inactivate BMP4 on the untreated PS surface. On the untreated PS surface, the 0.1, 0.2, and 2.5 ng/ul BMP4 droplets partially or fully inhibited cell attachment on the patterned area. Staining for laminin, a major component of Matrigel® matrix, after placing the 2.5 ng/ul BMP4 droplet on the untreated PS surface showed limited Matrigel® coating on the patterned region (FIG. 9B). This is likely due to the strong hydrophobic interaction between BMP4 and the surface that can have saturated the surface, preventing Matrigel® coating and cell attachment. Analysis was restricted to TC-treated PS surfaces because they permitted robust cell attachment on morphogen-patterned surfaces.

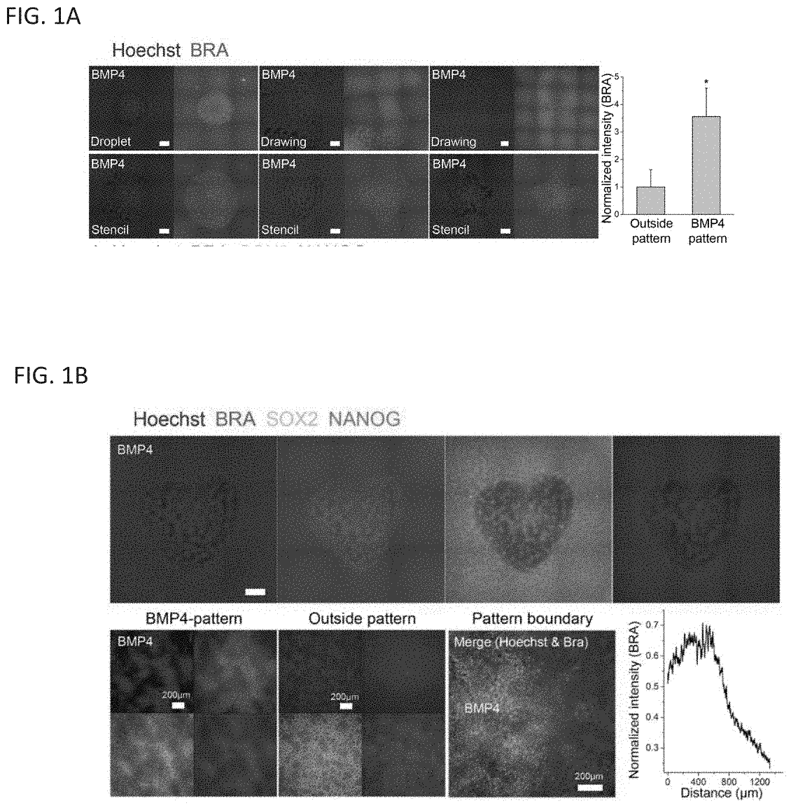

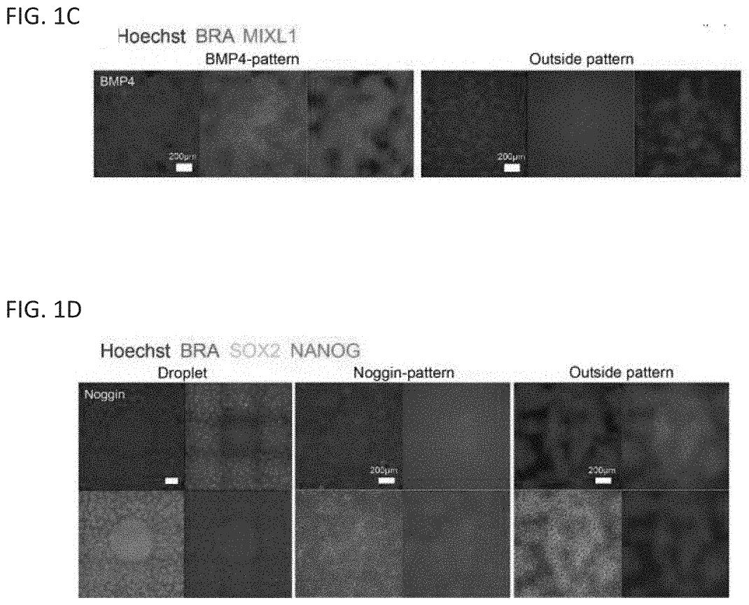

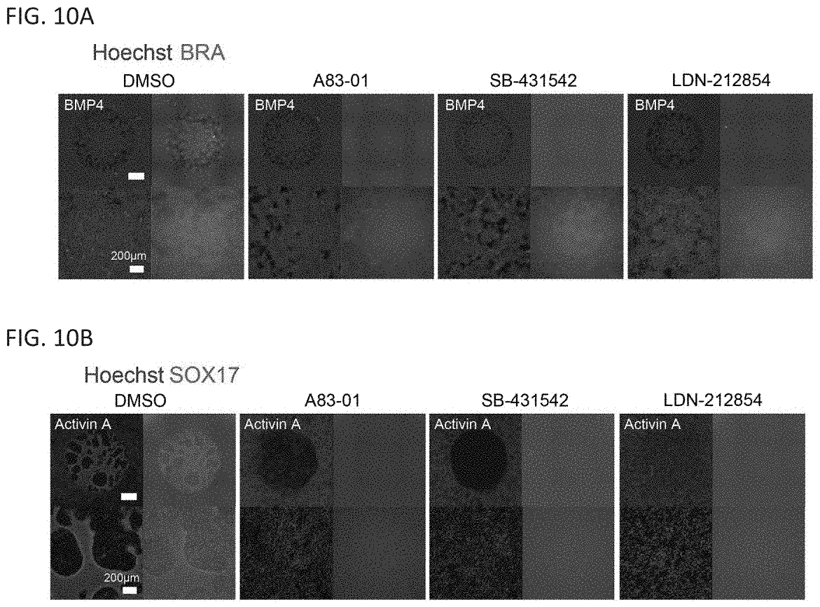

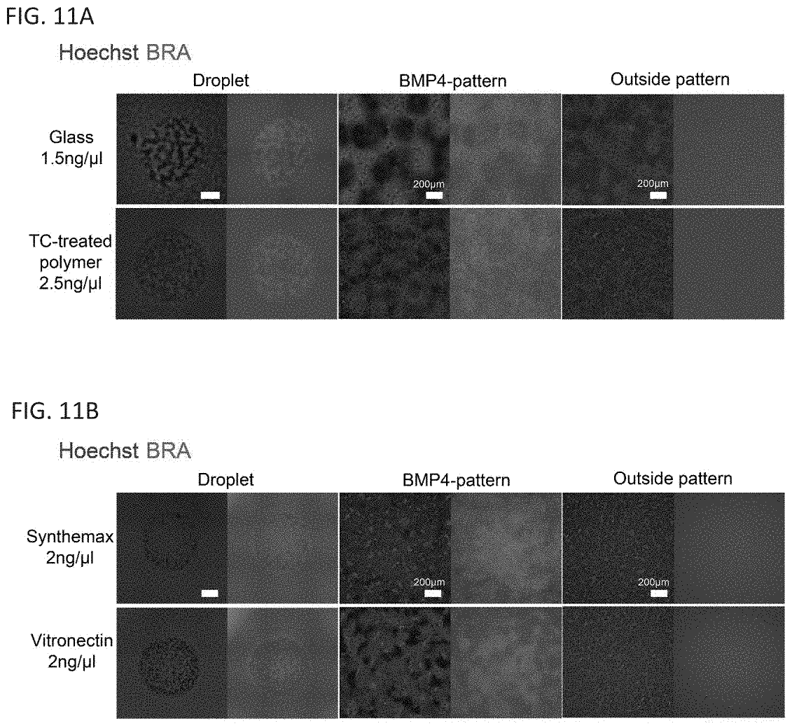

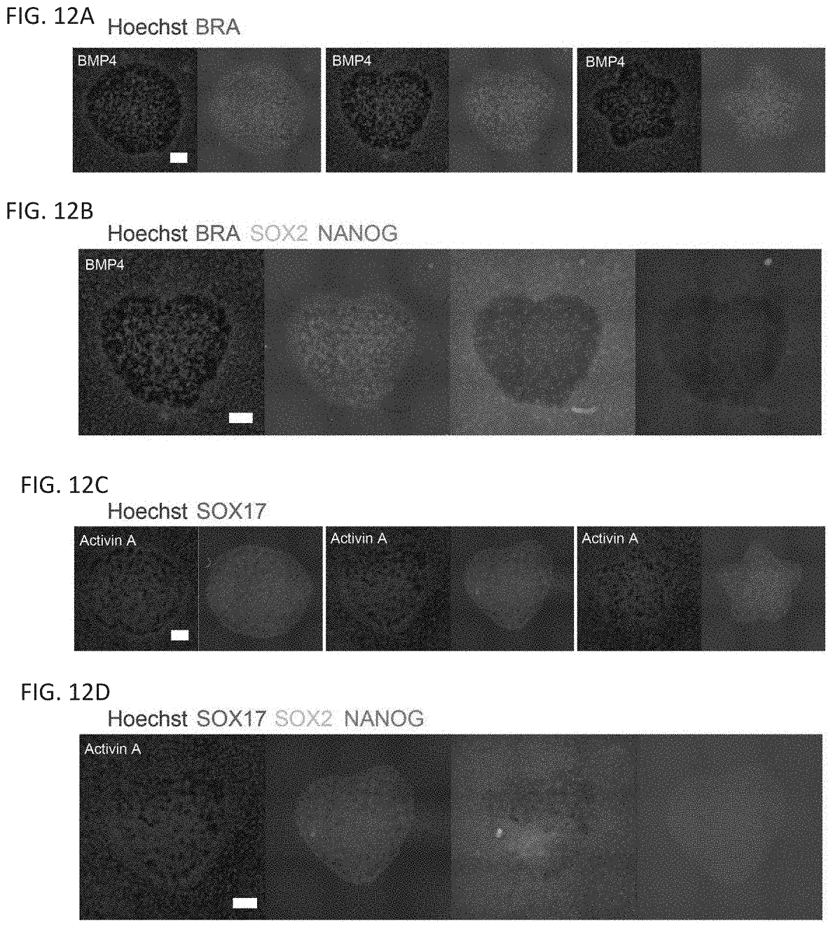

Next, BMP4 solution was placed on TC-treated plates in various patterns (droplet, letters, circle, heart, and star), and hPSCs seeded onto the substrate in mTeSR1 maintenance medium. Immunostaining for BRA after 2 days of culture demonstrated that localized BMP4 patterns led to spatially confined mesendoderm differentiation (FIG. 1A). Staining for the pluripotency markers SOX2 and NANOG showed that cells outside the BMP4-patterned regions maintained their pluripotent state, while the BRA+ mesendoderm cells expressed these pluripotency markers at a lower level than cells outside the BMP4 patterns (FIG. 1B). Some expression of pluripotency markers in the mesendoderm cells was expected according to previous studies (Yu et al. (2011) Cell stem cell 8, 326-334; Thomson et al. (2011) Cell 145, 875-889; Wang et al. (2012) Cell stem cell 10, 440-454). The expression profile of BRA at the edge of the pattern exhibited a sharp boundary, similar to the profile of adsorbed BMP4 (FIG. 8B), suggesting that the immobilized BMP4 acted directly on the hPSCs (FIG. 1B). This primitive streak-like mesendoderm fate was further verified by expression of MIXL1 in cells on the BMP4 pattern (FIG. 1C). Since the presence of inhibitory morphogens is also important in developmental patterning, a BMP inhibitor, noggin, was evaluated for antagonism to localized BMP4-induced mesendoderm differentiation. A noggin solution was placed as a droplet on TC-treated PS, then hPSCs were seeded and cultured in mTeSR1 supplemented with 10 ng/ml BMP4 for 2 days. Only the cells outside of the noggin pattern expressed BRA, while the cells on the noggin-treated patterned area did not express BRA (FIG. 1D). There results demonstrated that adsorbed noggin inhibited BMP4-induced mesendoderm differentiation. Small molecule inhibitors targeting different actin-receptor like kinases (ALKs) including A83-01 (inhibitor of ALK4, ALK5, and ALK7), SB431542 (inhibitor of ALK4, ALK5, and ALK7), and LDN-212854 (inhibitor of ALK2) were tested to examine whether receptor-level inhibition can antagonize the localized mesendoderm differentiation on BMP4 patterns (FIG. 10A). When these small molecules were added for the initial 24 h of culture on the patterned substrate, they suppressed BRA expression in the cells on the BMP4-patterned area, suggesting that localized mesendoderm differentiation required binding of BMP4 ligand and activation of corresponding receptors. This localized mesendoderm differentiation was also achieved on different substrates (glass and ibidi polymer coverslip) (FIG. 11A), different ECM coatings (Synthemax and vitronectin) (FIG. 11B), and with an additional hPSC cell line (FIG. 12A-FIG. 12B) demonstrating the versatility of the approach. Collectively, these data showed that this simple and robust strategy to localize morphogens such as BMP4 and noggin can spatially regulate BMP4 signaling in hPSC cultures, thereby resulting in localized mesendoderm differentiation.

### Example 4: Localized Differentiation of hPSCs into Definitive Endoderm by Patterned Activin a

A. Endoderm Differentiation

For localized definitive endoderm differentiation, 25 ng/μl activin A solution was placed with various patterns on a TC-treated plate. Matrigel® coating and hPSC seeding was performed as the same way described above. Medium was changed daily with RPMI1640 medium supplemented with 2% FBS. For the ALK inhibitor test, 1 μM A83-01 (Tocris, 2939), 10 μM SB-431542 (Tocris, 1614), or 2 μM LDN-212854 (Tocris, 6151) was added to mTeSR1 at day 0. At day 3, cells were immunostained for analysis.

B. Differentiation of hPSCs by Patterned Activin a

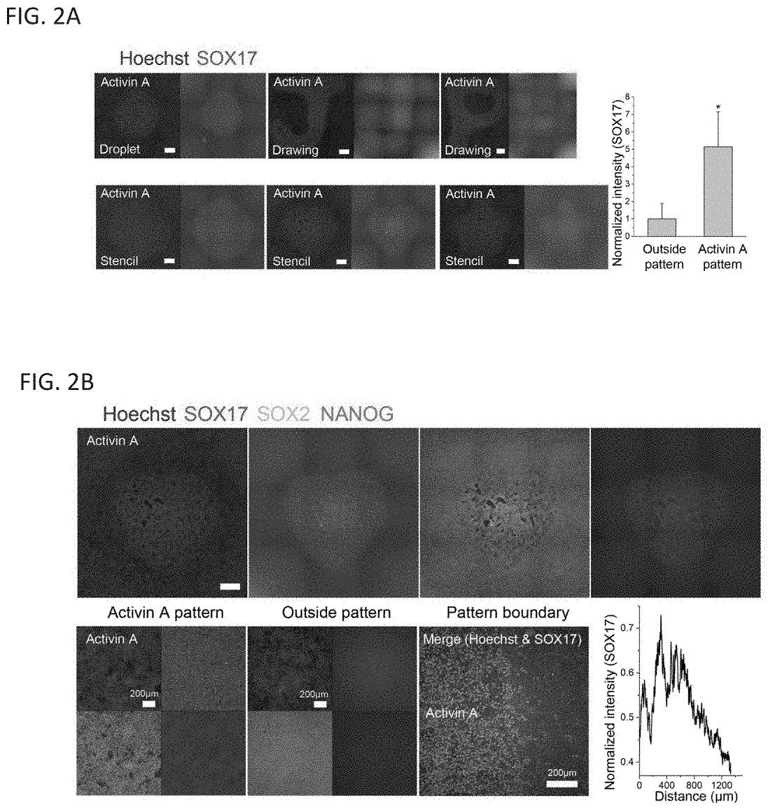

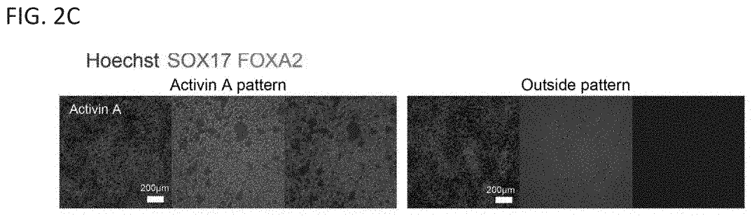

Since activin A has been reported to induce differentiation of hPSCs into definitive endoderm in the presence of a serum supplement (D'Amour et al. (2005) Nature Biotech. 23, 1534-1541), activin A was patterned as a way to direct localized endoderm commitment of hPSCs. Activin A solution was placed on TC-treated plate in a multitude of different patterns, similar to the BMP4 patterning described above. hPSCs were seeded on the activin A patterned substrates after coating with Matrigel, cultured for 3 days, and immunostained for SOX17, a marker for definitive endoderm (D'Amour et al. (2005) Nature Biotech. 23, 1534-1541). As with BMP4-induced mesendoderm differentiation, adsorbed activin A induced localized endoderm differentiation indicated by patterned expression of SOX17 (FIG. 2A). Immunostaining for NANOG and SOX2 showed that cells outside the activin A-patterned region retained SOX2 expression but lost NANOG expression, whereas cells on the activin A-patterned region expressed both SOX2 and NANOG as well as SOX17 (FIG. 2B). The retained expression of NANOG and SOX2 in the SOX17+ endoderm population was consistent with the role of these pluripotency factors in directing early endoderm specification (Teo et al. (2011) Genes and Development 25, 238-250). The SOX2+/NANOG-population found outside of the activin A patterned region is likely of ectoderm lineage because expression of SOX2 is maintained during ectodermal differentiation (Thomson et al. (2011) Cell 145, 875-889; Wang et al. (2012) Cell stem cell 10, 440-454). These cells were cultured in RPMI1640 basal medium supplemented with fetal bovine serum (FBS), which presumably induces spontaneous differentiation of hPSCs. Similar to the BMP4-induced BRA expression profile, the expression profile of SOX17 at the edge of the pattern showed a sharp boundary, suggesting that activin A acted directly on the cells in contact with the patterns (FIG. 2B). Small molecule-mediated ALK receptor inhibition abolished the patterned endoderm differentiation, again showing that differentiation is induced via the binding of the activin A to the receptors and activation of these receptors (FIG. 10B).

Localized patterning of the definitive endoderm was also achieved in WTC11 iPSCs (FIG. 12C-FIG. 12D). Since it has been reported that SOX17 also plays an important role in the development of mesoderm lineages such as cardiac (Liu et al. (2007) PNAS 104, 3859-3864), hematopoietic (Clarke et al. (2013) Nature cell biology 15, 502-510), and vascular tissue (Lange et al. (2014) Developmental Biology 387, 109-120), the cells that activin A patterns were verified to be definitive endoderm with an additional marker, FOXA2. At day 5 of differentiation, almost all of the cells on the activin A-patterned area expressed both SOX17 and FOXA2, while virtually no cells outside of the activin A patterns expressed either of these markers (FIG. 2C). Taken together, these results showed that localized mesendoderm and endoderm differentiation can be accomplished by patterning BMP4 and activin A adsorption on a substrate, respectively.

### Example 5: Patterned BMP4 and Activin a in a Single Well

A. Materials and Methods: Multi-Lineage Differentiation

For multi-lineage differentiation, 2.5 ng/μl BMP4 and 25 ng/μl activin A solution were placed in a heart-shaped and a star-shaped hole, respectively, of PDMS masks in a single well of a 12-well TC-treated plate. Matrigel® coating and hPSC seeding was performed as the same way described above. Medium was changed daily with RPMI1640 medium supplemented with 2% FBS. At day 3, 5, and 7, cells were immunostained for analysis.

B. Localized Multi-Lineage Differentiation of hPSCs by Patterned BMP4 and Activin a in a Single Well

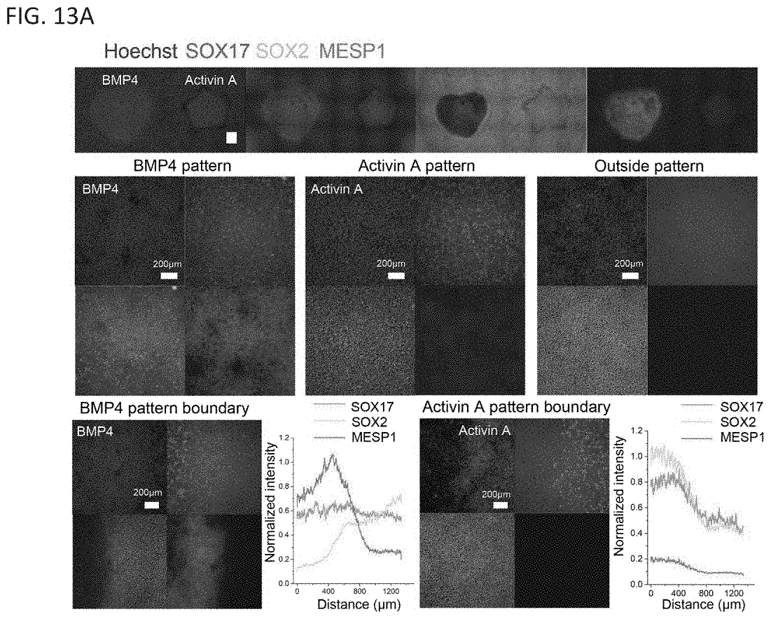

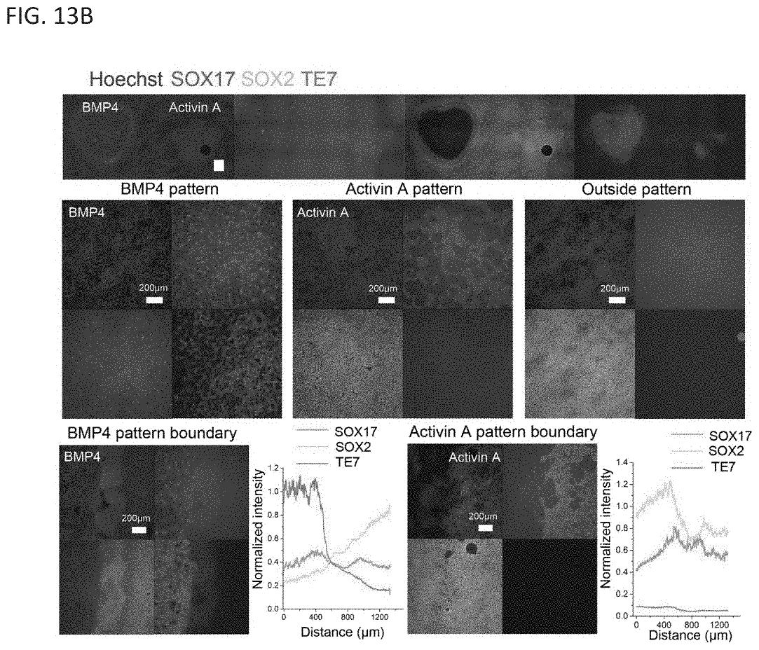

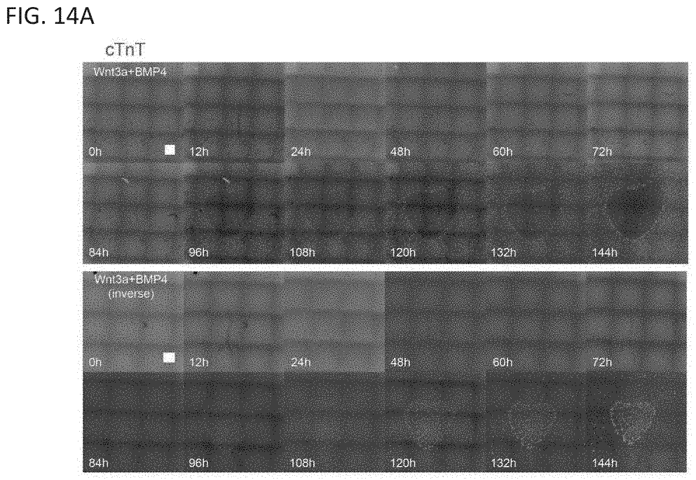

To examine whether the versatility of spatially directing hPSC differentiation by patterning morphogens would permit spatial control of hPSC differentiation to multiple germ layers in a single culture, BMP4 and activin A solutions were placed in PDMS stencils in different regions of single well, then hPSCs were seeded and cultured in these wells. At day 3 of differentiation, the cells on the BMP4-patterned areas expressed MESP1, a mesodermal transcription factor that regulates cardiac, hematopoietic, and skeletal myogenic specification (Chan et al. (2013) Cell stem cell 12, 587-601), and lost expression of the pluripotency and ectodermal marker SOX2 (FIG. 13A). Some of the cells on the BMP4 patterns expressed low levels of SOX17, which suggested the presence of endoderm cells derived from the BMP4-induced mesendoderm population or mesoderm cells that transiently expressed SOX17 (Liu et al. (2007) PNAS 104, 3859-3864). The cells on the activin A-patterned region expressed SOX17 but not MESP1. At day 5 of differentiation, TE7 antibody, which is raised against human thymic stroma (Haynes et al. (1984) The Journal of experimental medicine 159, 1149-1168) and has been used to detect mesoderm derived stromal cells such as fibroblasts, was used as a marker for mesoderm lineage cells. The cells on the BMP4-patterned region were stained by TE7 but did not express SOX2 (FIG. 13B). Some of the cells on the BMP4 patterns at day 5 also expressed SOX17, like the cells at day 3. The cells on the activin A-patterned area expressed SOX17 and SOX2, but not TE7.

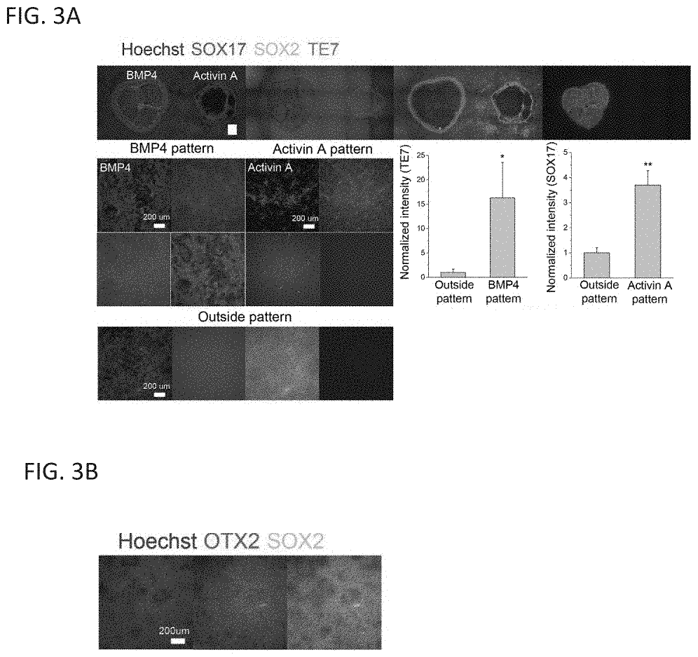

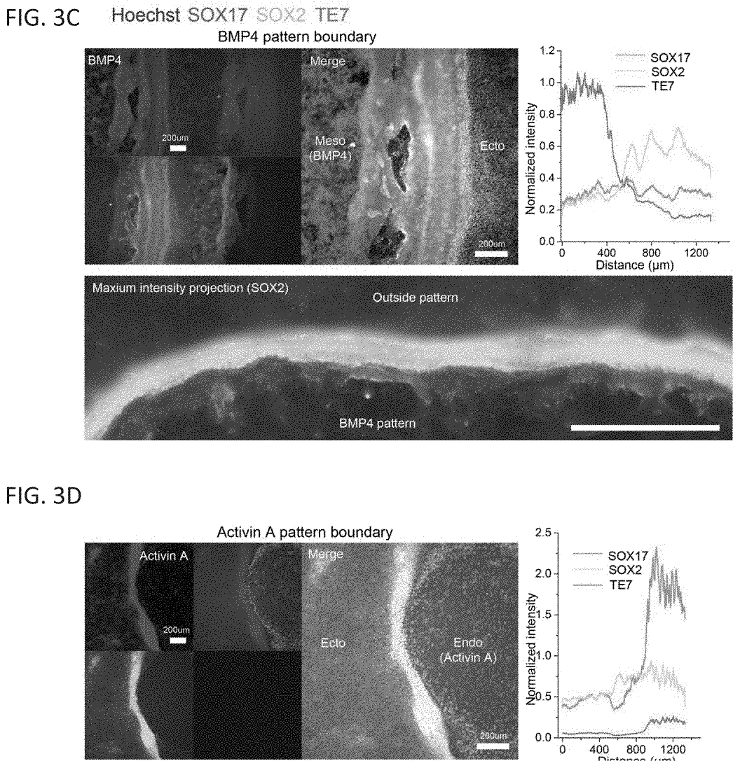

To further investigate the identity of these cells on the BMP4 and activin A patterns immunostaining at day 7 was performed. Significantly, most of the cells on the BMP4-patterned region lost expression of SOX17 but stained positive with TE7, while the cells on the activin A-patterned region lost expression of SOX2 but retained expression of SOX17 (FIG. 3A). The SOX2+ SOX17− TE7− cells outside the morphogen-patterned regions are likely a spontaneously differentiated ectoderm lineage population, as these cells were cultured in serum-containing medium for 6 days. Staining for the ectodermal transcription factor OTX2 further verified the ectoderm fate of the cells outside the morphogen-patterned regions (FIG. 3B). Another interesting characteristic of this patterned differentiation culture was the very dense and volumetric (˜200 μm thick) layer of SOX2+ SOX17- TE7- cells that was observed at the BMP4 pattern boundary, directly adjacent to the mesodermal-derived cells (FIG. 3C). A maximum intensity projection image of multiple z-slices for SOX2 showed a clear difference between this dense layer and the monolayer of ectodermal cells more distal to the morphogen patterns. Since this layer was only observed at the interface between the mesoderm linage cells and the ectoderm lineage cells, it suggested that the differentiating mesodermal cells could exert signaling to affect proliferation or differentiation fate of the cells in the surrounding layer through juxtacrine or localized paracrine interactions. Taken together, these data demonstrated spatially controlled differentiation of hPSC into the three germ layers in a single well and suggested the importance of crosstalk between developing germ layers (FIG. 3A-FIG. 3D).

### Example 6: Localized Differentiation of hPSCs into Cardiomyocytes by Patterned BMP4

For localized cardiomyocyte (CM) differentiation, 2.5 ng/μl BMP4 solution was placed in PDMS stencil masks on a TC-treated plate. Matrigel® coating and hPSC seeding was performed as described above. At day 1 and day 2, medium was changed with fresh RPMI B27−. Afterwards, CM differentiation was performed based on the GiWi protocol (Lian et al. (2013) Nature protocols 8, 162-175). At day 3, cells were treated with 5 μM IWP2 in fresh RPMI B27−. At day 5, medium was changed with fresh RPMI B27−. At day 7, medium was changed with fresh RPMI B27+. At day 8, a beating phenotype of patterned CM derived from H9-hTNNT2-GFP hESC was recorded using a fluorescence microscope (Nikon, Eclipse Ti2-E) equipped with a heating stage top incubator (Tokai Hit, Tokai Hit STX Series Stage Top Incubator System). Cells were also immunostained for analysis at day 8.

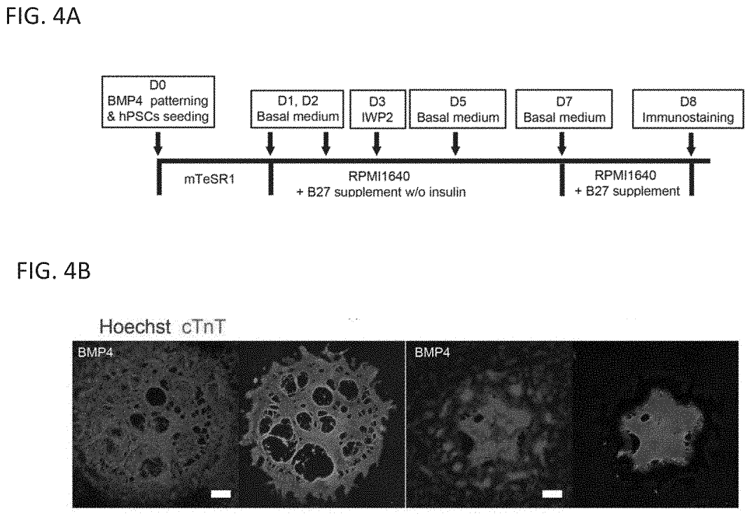

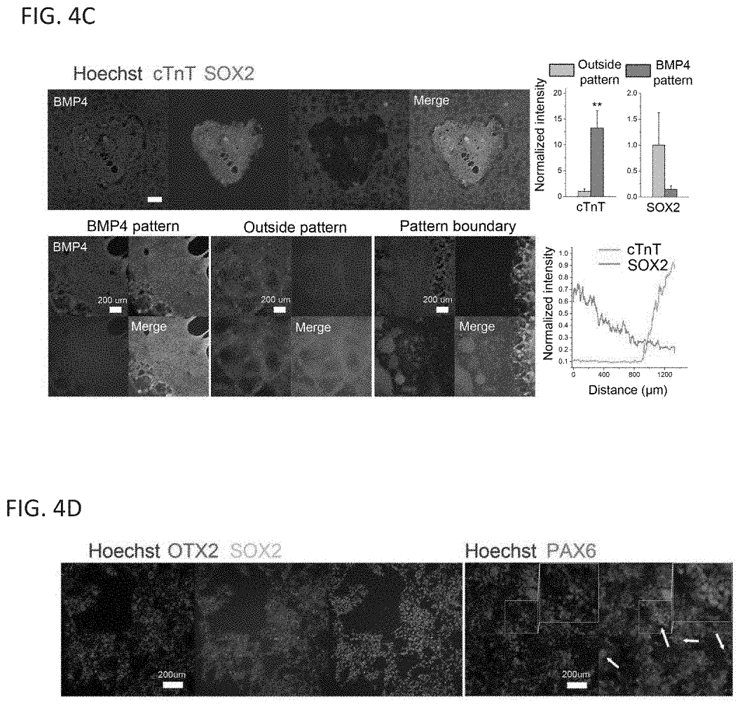

Based on the results showing localized mesoderm differentiation on BMP-patterned substrates, mesodermal cells were examined to see if they could be further differentiated on these patterns into cardiomyocytes, a specialized mesodermal cell type. BMP4 patterns were used to direct mesoderm and Wnt inhibition by addition of IWP2 to the culture medium at day 3 to specify cardiac mesoderm and achieve patterned CM differentiation (FIG. 4A). The H9-hTNNT2-GFP hESC reporter line was used to visualize patterning of CMs in live cells. At day 8 of differentiation, immunostaining of the CM-specific marker, cardiac troponin T (cTnT), showed localization of CMs to the patterned area in H9 hESCs (FIG. 4B) and live videos of the H9-hTNNT2-GFP line showed spontaneous contraction of patterned CMs. Cells outside of the BMP4 patterns did not express cTnT but retained SOX2 expression (FIG. 4C). Since these cells did not receive cues to undergo mesoderm differentiation, they likely spontaneously differentiated to neuroectoderm in the RPMI 1640 medium supplemented with B27, which has been reported to support neuronal induction and maturation (Nicholas et al. (2013) Cell stem cell 12, 573-586). Staining for OTX2 verified the ectoderm fate of the cells outside of the BMP4 patterns (FIG. 4D). Nuclear expression of PAX6 in some of these cells showed the presence of neuroectoderm cells in this population (FIG. 4D). These differentiation results supported the conclusion that the initial localized patterning of hPSCs into specific germ lineages can further be propagated toward terminally differentiated cell types while maintaining spatial patterns.

### Example 7: Localized Differentiation of Cardiac Progenitor Cells (CPCs) into Epicardial Cells by Patterned Wnt3a and BMP4

A. EpiC Differentiation

For localized EpiC differentiation, BMP4 (2 ng/μl)+Wnt3a (40 ng/μl) solution was placed in PDMS stencil masks either on a TC-treated plate or an ibidi 35 mm polymer dish. Matrigel® coating was performed as described above. Cryopreserved CPCs were thawed and seeded at 500,000-550,000 cells/cm² in LaSR medium (advanced DMEM/F12 (Gibco, 12634-010) supplemented with 60 μg/ml ascorbic acid (Sigma, A8960) and 2.5 mM Glutamax (Gibco, 35050-061)) supplemented with 5 μM Y-27632). Medium was changed daily with fresh LaSR medium until analysis. Time lapse imaging of CPCs derived from H9-hTNNT2-GFP hESC during localized EpiC differentiation was performed using a fluorescence microscope equipped with a heating stage top incubator. 18 days after CPC seeding, cells cultured on an ibidi polymer dish were immunostained and imaged using a confocal microscope (Nikon, A1R-SI+) for the analysis of sarcomere structure.

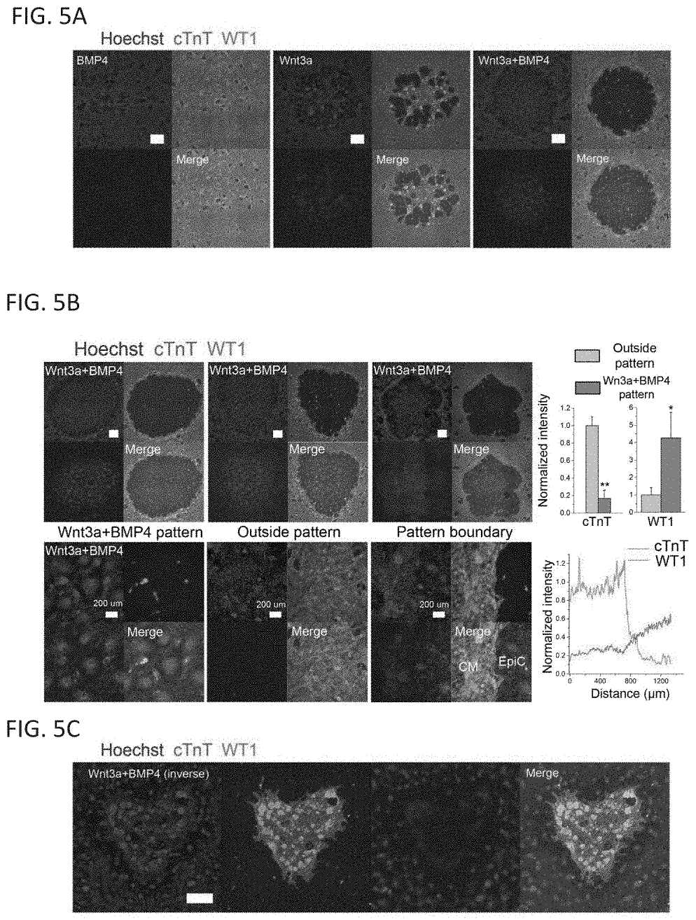

Spatial organization of cell fate commitment is important throughout development. To mimic later stage cell fate patterning, the same patterned morphogen approach for the localized germ layer specification of hPSCs was examined to see if it could be applied to hPSC-derived cardiac progenitors. hPSC-derived cardiac progenitors possessing potential to be differentiated into CMs and EpiCs were generated by the GSK inhibitor and Wnt inhibitor (GiWi) protocol (Bao et al. (2016) Nature biomed. Eng. 1, 1-12). These cardiac progenitors can further differentiate into CMs without any specific external signaling factors and can also differentiate into EpiCs upon activation of canonical Wnt signaling (Bao et al. (2016) Nature biomed. Eng. 1, 1-12). Since previous studies showed that both BMP4 and Wnt3a play important roles in the generation of EpiCs (Bao et al. (2016) Nature biomed. Eng. 1, 1-12; Iyer et al. (2015) Development 142, 1528-1541), droplets containing BMP4, Wnt3a, or both BMP4 and Wnt3a were placed on a TC-coated PS plate and hPSC-derived cardiac progenitors were seeded on these patterns to assess localized EpiC differentiation. While BMP4 and Wnt3a individually induced limited if any EpiC differentiation, the droplet containing both BMP4 and Wnt3a successfully induced localized EpiC differentiation (FIG. 5A) indicated by the expression of WT1, a transcription factor expressed in epicardial cells. This result was consistent with a previous study showing the synergistic effects of Wnt3a and BMP4 on EpiC specification (Iyer et al. (2015) Development 142, 1528-1541) and demonstrated that multiple morphogens can be patterned on the same region to have a collaborative effect on localized differentiation.

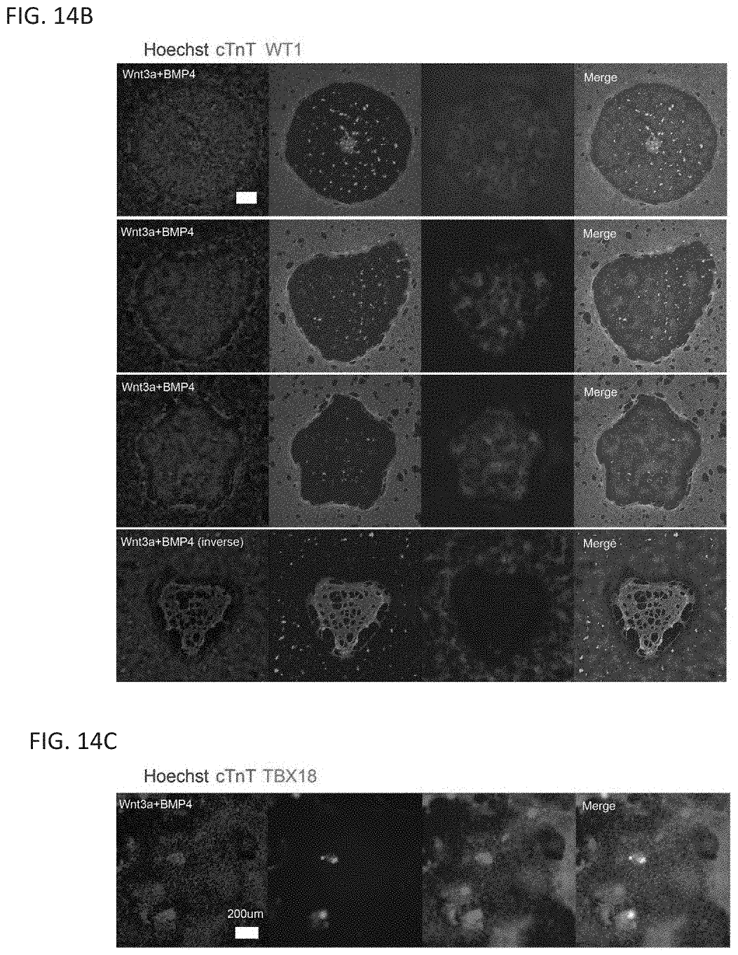

Using PDMS stencils, different EpiC patterns were created in which the EpiCs were generated on Wnt3a+BMP4 patterns while CMs differentiated outside of these patterned regions (FIG. 5B). Using a PDMS fragment to exclude Wnt3a+BMP4 solution from contacting the substrate generated a heart-shaped CM population surrounded by EpiCs (FIG. 5C). Time-lapse imaging of cardiac progenitors generated from H9-hTNNT2-GFP hESCs showed that the CM pattern formation was observed beginning at day 4 after cardiac progenitor seeding (FIG. 14A). This cell line also exhibited localized EpiC-CM differentiation with different patterns (FIG. 14B). Epicardial cell fate was further confirmed by expression of TBX18 in the cells on Wnt3a+BMP4 pattern (FIG. 14C). Taken together, these data demonstrated that localized differentiation via morphogen patterning can be applied to more committed progenitor cells and, with the choice of appropriate morphogens, cell types such as CMs and EpiCs can be spatially patterned on a substrate.

### Example 8: Effects of Epicardial Cells on Cardiomyocytes in a Spatially Organized Context

The epicardium and the myocardium actively interact with each other during heart development. It has been reported that EpiCs or epicardial-derived cells affect proliferation and maturation of CMs (Weeke-Klimp et al. (2010) Journal of molecular and cellular cardiology 49, 606-616; Bargehr et al. (2019) Nature biotechnology 37, 895-906). Mouse CMs exhibited cellular elongation and alignment when cocultured with epicardial-derived cells obtained from proepicardial organ explant in vitro (Weeke-Klimp et al. (2010) Journal of molecular and cellular cardiology 49, 606-616). Additionally, hESC-derived CMs exhibited longer sarcomere length when cultured with hESC-derived EpiCs in 3D engineered heart tissue (3D-EHT) in vitro and when transplanted with hESC-derived EpiCs as the 3D-EHT in vivo (Bargehr et al. (2019) Nature biotechnology 37, 895-906). This suggested important crosstalk mechanisms between EpiCs in altering CM phenotype.

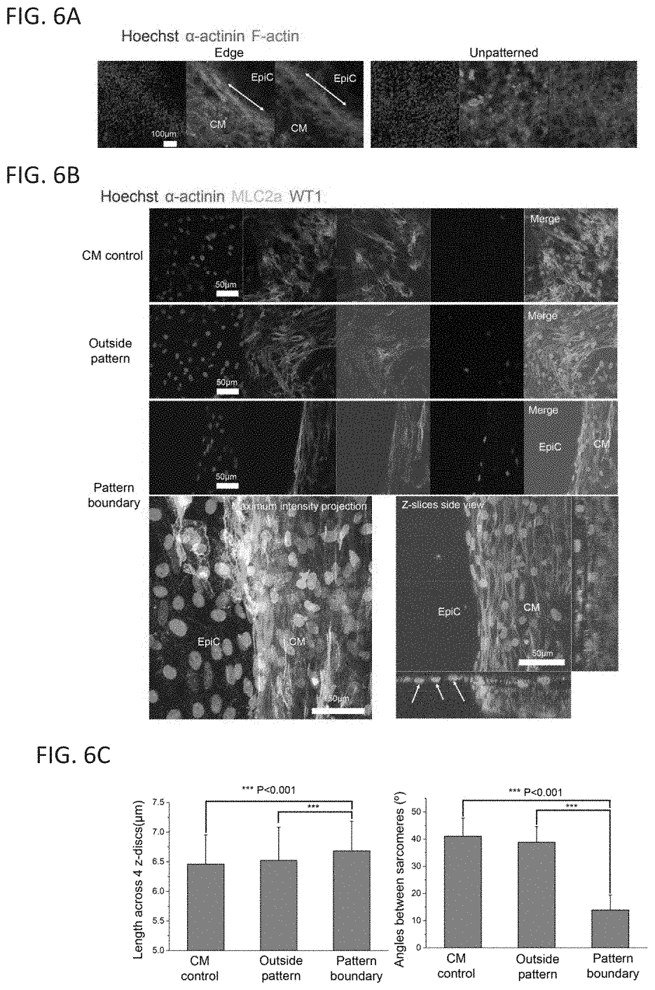

EpiC and CM patterned cells were stained for α-actinin and F-actin to enable analysis of sarcomere and myofilament structures in CMs in order to observe EpiC-CM crosstalk response in a spatially controlled differentiation co-culture. Epifluorescence images showed a stark difference in α-actinin and F-actin features between CMs in contact with EpiCs and CMs distal from the EpiC pattern. CMs located at the edge of the EpiC pattern had more aligned myofibers, suggestive of enhanced structural maturation (FIG. 6A). Confocal microscopy of α-actinin with MLC2a and WT1 for identification of CMs and EpiCs, respectively, showed more aligned sarcomeric α-actinin in CMs at the edge of the EpiC pattern compared to CMs on the outside of the patterns and a CM monoculture control (FIG. 6B). A maximum intensity projection image of the multiple z-slices at the edge of the pattern and the side view of these z-slices showed an EpiC monolayer on the patterned region and several EpiCs under the volumetric CM layers. Quantification of the distance between α-actinin+z-lines and the angles between these z-disc-crossing lines showed that the CMs located adjacent to EpiCs exhibited longer sarcomere length and increased myofibril alignment than CMs away from the boundary. Given that sarcomere length is an indicator of CM structural maturation and myofibril alignment of CMs is an important feature of CMs in vivo (Bursac et al. (2002) Circulation research 91), these results suggested that the positive effects of EPIs on the structural maturation of hPSC-derived CMs occurred over a short length scale.

In summary, these experiments demonstrated that localized differentiation on morphogen patterns can be used to systematically derive spatially distinct cell populations and to assess cell-cell interactions in differentiating cell populations in a spatial context.

While particular aspects and embodiments are disclosed herein, other aspects and embodiments will be apparent to those skilled in the art in view of the foregoing teaching. The various aspects and embodiments disclosed herein are for illustration purposes only and are not intended to be limiting, with the true scope and spirit being indicated by the following claims.

What is claimed is:

1. An in vitro method for differentiating pluripotent stem cells, the method comprising:
a) patterning one or a plurality of signaling proteins on a portion of a suitable surface;
b) coating the suitable surface comprising the one or a plurality of signaling proteins with a cell culture substrate; and
c) plating a suspension of the pluripotent stem cells on the suitable surface coated with the cell culture substrate.

2. The method of claim 1, wherein the solution of pluripotent stem cells is plated at a concentration of about 5×10⁵ to about 1×10⁶ cells/cm′.

3. The method of claim 1, wherein the cell culture substrate is Matrigel®, vitronectin, or laminin.

4. The method of claim 3, wherein the cell culture substrate is Matrigel®.

5. The method of claim 1, wherein the one or a plurality of signaling proteins are patterned on the suitable surface at a concentration of 1 ng/μ1 to about 1 μg/μ1.

6. The method of claim 1, wherein the suitable surface is hydrophilic.

7. The method of claim 6, wherein the suitable surface comprises a tissue-culture treated polystyrene plate.

8. The method of claim 1, wherein the pluripotent stem cells are differentiated into one or more of mesoderm, endoderm, ectoderm, cardiomyocytes or epicardial cells.

9. The method of claim 1, wherein the one or a plurality of signaling proteins are placed on the suitable surface in a geometric pattern.

10. The method of claim 9, wherein the pattern of signaling proteins is created on the suitable surface using a polydimethylsiloxane (PDMS) stencil cut into a desired shape.

11. The method of claim 9, wherein the one or a plurality of signaling proteins are patterned in a concentration gradient.

12. The method of claim 1, wherein the one or a plurality of signaling proteins are a morphogen.

13. The method of claim 1, wherein the one or a plurality of signaling proteins is BMP4, activin A, noggin, or Wnt3a.

14. The method of claim 1, wherein the pluripotent stem cells are human embryonic stem cells (hESCs) or human induced pluripotent stem cells (hiPSCs).
